(19) United States
(12) Patent Application Publication
(10) Pub. No.: US 2024/0402069 A1
(43) Pub. Date: Dec. 5, 2024

(54) CORROSION ESTIMATION DEVICE AND METHOD

(71) Applicant: Nippon Telegraph and Telephone Corporation, Tokyo (JP)

(72) Inventors: Shingo Mineta, Tokyo (JP); Shota Oki, Tokyo (JP); Mamoru Mizunuma, Tokyo (JP); Masayuki Tsuda, Tokyo (JP)

(21) Appl. No.: 18/692,233

(22) PCT Filed: Oct. 29, 2021
(86) PCT No.: PCT/JP2021/039982

§ 371 Date: Mar. 14, 2024

(51) Int. Cl. G01N 17/00; G01N 33/24

(52) U.S. Cl. CPC G01N 17/00; G01N 33/246

(57) ABSTRACT

The corrosion estimation device includes a sensor, a first processing circuit, a second processing circuit, and a third processing circuit. The sensor measures a water content of soil of a target land. The first processing circuit estimates a damping function representing a temporal change in a ratio of an area wetted with water on a surface of a metal structure buried in the target land on the basis of a water content measurement value measured by the sensor. The second processing circuit estimates, from the estimated damping function, a rate increase function that increases the corrosion rate of the metal structure, the rate increase function being derived from a thickness of a water film formed on the surface of the metal structure. The third processing circuit estimates a temporal change function of the corrosion rate given by a product of the damping function and the rate increase function.

### CROSS-REFERENCE TO RELATED APPLICATIONS

This application is a national phase entry of PCT Application No. PCT/JP2021/039982, filed on Oct. 29, 2021, which application is hereby incorporated herein by reference.

### TECHNICAL FIELD

The present invention relates to a corrosion estimation device and a corrosion estimation method for estimating corrosion of a structure buried in soil.

### BACKGROUND

There are many types of infrastructure equipment that support our life, and the number of pieces of infrastructure equipment is also enormous. In addition, infrastructure equipment is exposed to various environments not only in urban areas but also in mountainous areas, the vicinity of coasts, hot spring areas, cold areas, and the sea and the ground, and deterioration forms and deterioration progress rates are various. For this reason, it is necessary to grasp a deterioration state of equipment by visual inspection or the like and to appropriately perform maintenance. However, in metal underground equipment represented by steel pipe columns, support anchors, steel pipes, and the like, a portion hidden in the ground cannot be visually inspected, and the maintenance according to the deterioration state is difficult.

Due to contact with soil, the metal underground equipment corrodes and deteriorates at different rates depending on external environments (Non Patent Literature 1, Non Patent Literature 2, and Non Patent Literature 3). There is a following method for determining a corrosion rate of a metal in soil. For example, the metal buried for a certain period is taken out, and the corrosion rate can be obtained by dividing a directly measured corrosion amount by a buried period. However, this method requires a long period of time to obtain the corrosion rate, and requires burying and excavation of a sample, removal of rust of the sample taken out, and the like, and work efficiency is significantly low. In addition, in this method, only a state change before burying and a state change after taking out can be obtained as information, and it is not possible to monitor a temporal change in the corrosion rate.

On the other hand, there is also a method of evaluating the corrosion rate by an electrochemical method such as an alternating current impedance method. In the alternating current impedance method, a charge transfer resistance Rct in soil is measured by applying a minute alternating current to an electrode. Since a reciprocal of the charge transfer resistance Rct is proportional to the corrosion rate, the corrosion rate can be evaluated by measuring the charge transfer resistance Rct. In addition, by monitoring the charge transfer resistance Rct, the temporal change in the corrosion rate can also be measured.

However, a measurement device used in the alternating current impedance method is generally expensive, and is not suitable for, for example, multi-point observation or long-term continuous measurement. Further, to measure a deep place in the ground, for example, a distance between the electrode and the measurement device becomes long, and there is also a problem of a difficulty in measurement due to an influence of external noise or the like.

### CITATION LIST

### Non Patent Literature

Non Patent Literature 1: Morio Kadoi et al., “Studies on Soil Corrosion of Metallic Materials (Part 1)—Fundamental Experiment on Soils—”, CORROSION ENGINEERING, Vol. 16, No. 6, pp. 238-246, 1967.

Non Patent Literature 2: Yoshikazu Miyata and Shukuji Asakura, “Corrosion Monitoring of Metals in Soils by Electrochemical and Related Methods: Part 2—Estimation of the Corrosion Rate Based upon the Combination of a Number of Information and Proposals of the Systematic Evaluation Process—”, Zairyo-to-Kankyo (in Japanese) (Materials and Environments), Vol. 46, No. 10, pp. 610-619, 1997.

Non Patent Literature 3: Satomi Tsunoda and Tetsuro Akiba, “Some Problem for Evaluating Soil Aggressivity”, Corrosion Engineering, Vol. 36, No. 3, pp. 168-177, 1987.

### SUMMARY

### Technical Problem

As described above, the conventional methods have a problem of a difficulty in easily and inexpensively measuring a corrosion rate of metals buried in the ground.

Embodiments of the present invention have been made to solve the above problems, and an object thereof is to enable simple and inexpensive measurement of a corrosion rate of metals buried in the ground.

### Solution to Problem

A corrosion estimation method according to embodiments of the present invention includes: a measurement step of measuring a water content of soil of a target land; a first processing step of estimating a damping function representing a temporal change in a ratio of an area wetted with water on a surface of a metal structure buried in the target land on the basis of a water content measurement value measured in the measurement step; a second processing step of estimating, from the damping function, a rate increase function that increases a corrosion rate of the metal structure, the rate increase function being derived from a thickness of a water film formed on the surface of the metal structure; and a third processing step of estimating a temporal change function of the corrosion rate given by a product of the damping function and the rate increase function.

Further, a corrosion estimation device according to embodiments of the present invention includes: a sensor configured to measure a water content of soil of a target land; a first processing circuit configured to estimate a damping function representing a temporal change in a ratio of an area wetted with water on a surface of a metal structure buried in the target land on the basis of a water content measurement value measured by the sensor; a second processing circuit configured to estimate, from the damping function, a rate increase function that increases a corrosion rate of the metal structure, the rate increase function being derived from a thickness of a water film formed on the surface of the metal structure; and a third processing circuit configured to estimate a temporal change function of the corrosion rate given by a product of the damping function and the rate increase function.

### Advantageous Effects of Embodiments of Invention

As described above, according to embodiments of the present invention, the damping function is estimated on the basis of the water content measurement value, the rate increase function derived from the thickness of the water film is estimated from the damping function, and the temporal change function of the corrosion rate given by the product of the damping function and the rate increase function is estimated, so that the corrosion rate of the metal buried in the ground can be easily and inexpensively measured.

### BRIEF DESCRIPTION OF THE DRAWINGS

FIG. 1 is a configuration diagram illustrating a configuration of a corrosion estimation device according to an embodiment of the present invention.

FIG. 2 is a flowchart for describing a corrosion estimation method according to the embodiment of the present invention.

FIG. 3 is a configuration diagram illustrating a hardware configuration of the corrosion estimation device according to the embodiment of the present invention.

FIG. 4 is an explanatory diagram illustrating an image of estimation using the corrosion estimation device according to the embodiment of the present invention.

FIG. 5 is a characteristic chart illustrating a temporal change in a corrosion rate of a metal in soil measured by an alternating current impedance method.

FIG. 6A is a cross-sectional view schematically illustrating a temporal change of water on a surface of a metal structure 201 buried in soil.

FIG. 6B is a cross-sectional view schematically illustrating a temporal change of water on the surface of the metal structure 201 buried in soil.

FIG. 7 is a characteristic chart illustrating a change in wet area ratio on the surface of the metal structure buried in soil.

FIG. 8A is a cross-sectional view schematically illustrating a state of a water film 204 on the surface of the metal structure 201.

FIG. 8B is a cross-sectional view schematically showing a state where the water film on the surface of metal structure 201 is changed to a thinner water film 204a due to drainage.

FIG. 9 is a characteristic chart illustrating a temporal change in the corrosion rate due to a decrease in water film thickness on the surface of the metal structure buried in soil.

FIG. 10 is a characteristic chart illustrating an example of a temporal change model of the corrosion rate of a metal in soil.

FIG. 11 is a characteristic chart illustrating a state in which the model of the corrosion rate is fitted to measured values illustrated in FIG. 5.

### DETAILED DESCRIPTION OF ILLUSTRATIVE EMBODIMENTS

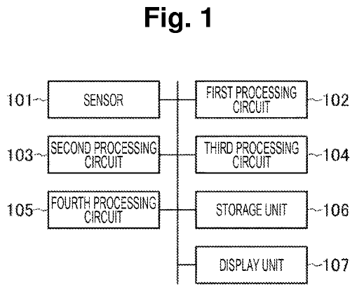

Hereinafter, a corrosion estimation device according to an embodiment of the present invention will be described with reference to FIG. 1. The corrosion estimation device includes a sensor 101, a first processing circuit 102, a second processing circuit 103, a third processing circuit 104, a fourth processing circuit 105, a storage unit 106, and a display unit 107.

The sensor 101 measures a water content of soil of a target land. The sensor 101 obtains the water content of the soil by, for example, measuring dielectric characteristics of the soil. In addition, the sensor 101 measures the water content of the soil using a matrix potential indicating a retention force of a soil moisture. A measured result can be stored in the storage unit 106.

The first processing circuit 102 estimates a damping function representing a temporal change in a ratio of an area wetted with water on a surface of a metal structure buried in the target land on the basis of a water content measurement value measured by the sensor 101. The estimated damping function can be stored in the storage unit 106.

The second processing circuit 103 estimates, from the estimated damping function, a rate increase function that increases the corrosion rate of the metal structure, the rate increase function being derived from a thickness of a water film formed on the surface of the metal structure. The second processing circuit 103 estimates the rate increase function from the damping function stored in the storage unit 106. The estimated rate increase function can be stored in the storage unit 106.

The third processing circuit 104 estimates a temporal change function of the corrosion rate given by a product of the damping function and the rate increase function. The third processing circuit 104 estimates the temporal change function of the corrosion rate from the damping function and the rate increase function stored in the storage unit 106.

The fourth processing circuit 105 obtains the corrosion rate of the metal structure from the measured water content of the soil of the target land using the temporal change function. In addition, the fourth processing circuit 105 displays the obtained corrosion rate on the display unit 107.

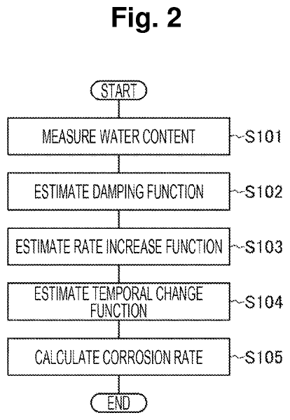

Next, a corrosion estimation method according to embodiments of the present invention will be described with reference to FIG. 2.

First, in step S101, the water content of the soil of the target land is measured by the sensor 101 (measurement step). Principles and means for measurement information of the soil water content of the target land are not limited as long as information of an amount of moisture or a proportion of the moisture contained in the soil can be obtained as the measurement information. Examples of a method include a method of deriving the measurement information by measuring dielectric characteristics of the soil, and a case of performing measurement using the matrix potential indicating a retention force of soil moisture. For example, the temporal change in soil water content is measured using a soil moisture sensor or the like.

Next, in step S102, the first processing circuit 102 estimates the damping function representing the temporal change in the ratio of the area wetted with water on the surface of the metal structure buried in the target land on the basis of the water content measurement value measured in step S101 (measurement step) (first processing step).

Next, in step S103, the second processing circuit 103 estimates the rate increase function that increases the corrosion rate of the metal structure, the rate increase function being derived from the thickness of the water film formed on the surface of the metal structure from the damping function (second processing step).

Next, in step S104, the third processing circuit 104 estimates the temporal change function of the corrosion rate given by the product of the damping function and the rate increase function (third processing step).

Next, in step S105, the fourth processing circuit 105 obtains the corrosion rate of the metal structure from the measured water content of the soil of the target land using the obtained temporal change function (fourth processing step). The obtained corrosion rate is displayed on the display unit 107.

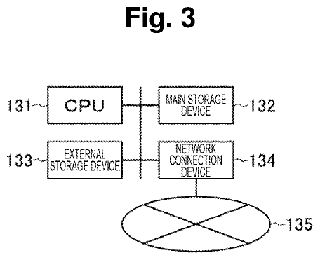

Note that, as illustrated in FIG. 3, the first processing circuit 102, the second processing circuit 103, the third processing circuit 104, the fourth processing circuit 105, and the storage unit 106 of the corrosion estimation device according to the above-described embodiment can implement the above-described functions (corrosion estimation method) by forming a computer device including a central processing unit (CPU) 131, a main storage device 132, an external storage device 133, a network connection device 134, and the like, and causing the CPU 131 to be operated by a program developed in the main storage device 132 (by executing the program). The above-described program is a program for the computer to execute the corrosion estimation method described in the above embodiment. The network connection device 134 is connected to a network 135. In addition, the functions can be distributed to a plurality of computer devices.

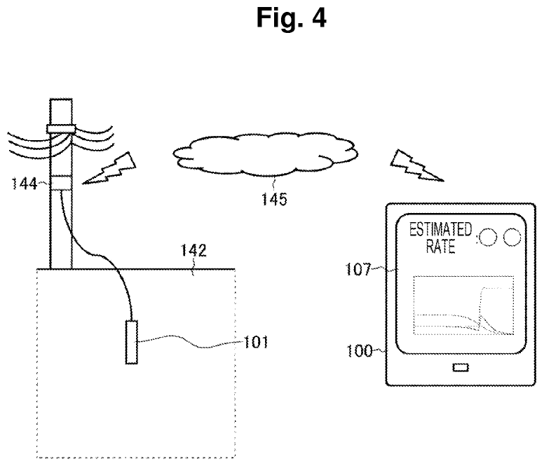

Here, as illustrated in FIG. 4, the water content of the soil of the target land can be obtained by installing the sensor 101 in soil 142 in which equipment that is a structure configured by metal to be estimated is buried. It is favorable to install the sensor 101 in an environment as close as possible to the installation environment of the equipment, and accuracy is enhanced by installing the sensor 101 as close as possible to the equipment.

Furthermore, a corrosion estimation device 100 can be a computer device as described above, and can be implemented by, for example, a general personal computer or an electronic device such as a tablet. For example, the sensor 101 may be provided with a transmission function 144 for transmitting measured information so as to be able to communicate with the corrosion estimation device 100 via a communication network 145. In addition, one corrosion estimation device 100 can correspond to the plurality of sensors 101. The display unit 107 can be implemented by a monitor of a personal computer, a wireless device, or the like.

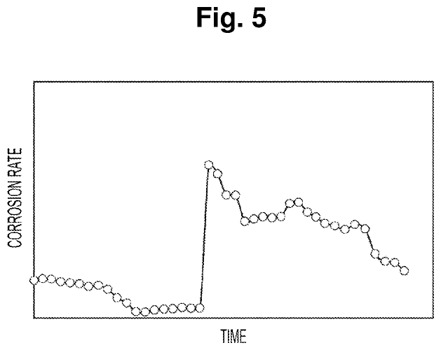

This will be described in more detail below. Corrosion in soil proceeds by a chemical reaction with the soil in contact with the surface of the metal structure. FIG. 5 is an example of a temporal change in a corrosion rate (1/Rct, Rct: Charge transfer resistance) of the metal in the soil measured by the alternating current impedance method. This is a typical example representing the change in the corrosion rate of when time=0 is occurrence of rainfall and drainage progresses over time after the rainfall ceases.

There is no significant change in the corrosion rate for a while after the drainage progresses, but the corrosion rate rapidly increases with the lapse of time and then turns to decrease. Although FIG. 5 illustrates the change in the corrosion rate due to one rainfall, it is known that the behavior of FIG. 5 is repeated every rainfall in actual soil.

As described above, the soil environment is characterized in that the amount of moisture in the soil changes over time due to rainfall, and the corrosion rate changes depending on states of the water on the surface of the buried metal structure and dissolved oxygen in the water. First, the water on the surface of the metal structure is considered. During rainfall, the soil is filled with water, but as the rain stops and the water drains, the moisture content (soil water content) in the soil decreases. Therefore, the amount of water present on the surface of the buried metal also changes over time.

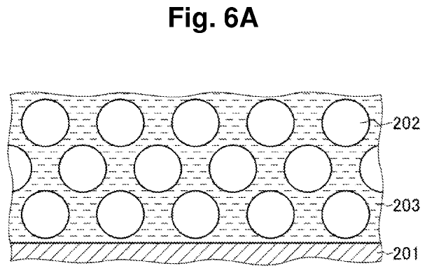

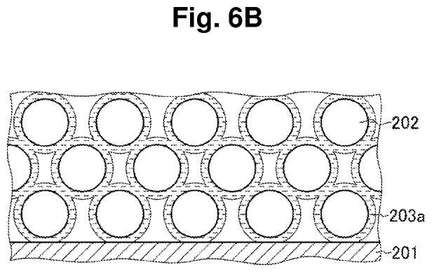

FIGS. 6A and 6B are schematic views illustrating the temporal change of the water on the surface of the metal structure 201 buried in soil. A plurality of soil particles 202 exists on the surface of the metal structure 201. Since corrosion of metals occurs in a portion in contact with water, a ratio of corrosion on the surface of the metal structure 201 is equal to a ratio of an area wetted with water. A state in which the soil is filled with water during rainfall is defined as a wet area ratio=1. In this state, as illustrated in FIG. 6A, a space between the adjacent soil particles 202 is filled with soil water 203. Thereafter, as the rain stops and the water drains, the soil water 203 filling the space between the adjacent soil particles 202 decreases, and as illustrated in FIG. 6B, a water film 203a is formed around each of the soil particles 202, a space without water is formed in the space between the adjacent soil particles 202, and the wet area ratio on the surface of the metal structure 201 decreases.

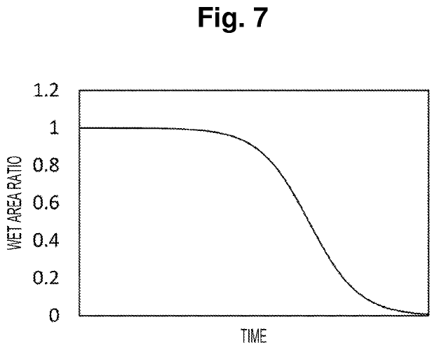

In general soil, drainage starts and water initially escapes into the ground according to gravity. Thereafter, the water trapped by a capillary phenomenon of the soil remains for a long time and is dried by evaporation or the like, so that the change in the wet area ratio exhibits a temporal damping behavior as illustrated in FIG. 7.

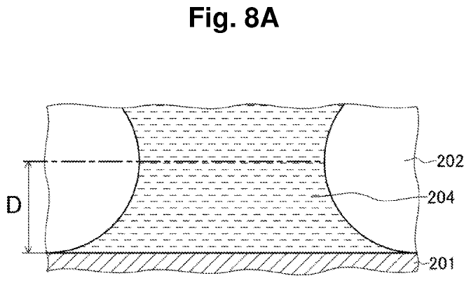

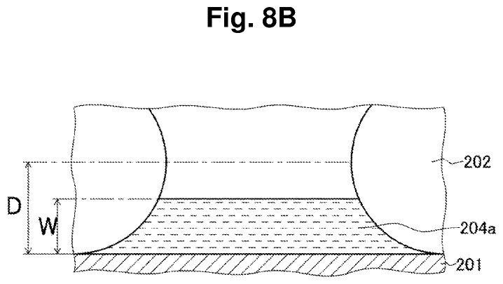

Meanwhile, an influence of the dissolved oxygen of water on the corrosion rate varies depending on the water film thickness on the surface of the metal structure. The water film thickness on the surface of the metal structure decreases with drainage. FIGS. 8A and 8B illustrate a state in which the thickness of the water film 204 on the surface of metal structure 201 associated with drainage changes to a thinner water film 204a.

Here, as illustrated in FIG. 8A, in a case where the thickness of the water film 204 existing on the surface of the metal structure 201 is equal to or larger than a steady diffusion layer thickness D, the corrosion rate is constant regardless of the water film thickness. Therefore, the influence of the water film thickness on the corrosion rate is substantially constant until drainage proceeds to some extent from the occurrence of rainfall.

Meanwhile, when the drainage proceeds to some extent and a thickness W of the water film 204a on the surface of the metal structure 201 becomes smaller than the steady diffusion layer thickness D, as illustrated in FIG. 8B, the corrosion rate increases as the water film thickness W becomes smaller.

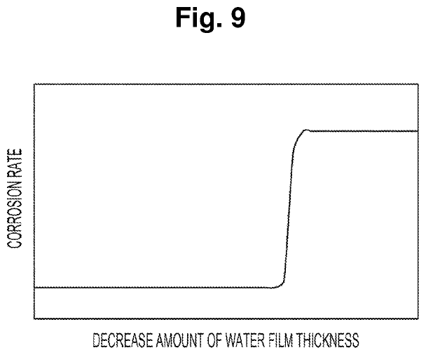

The thickness of the water film is related to diffusion-limiting current density of the dissolved oxygen, and is theoretically proportional to an oxygen partial pressure of a gas with which the thin water film is in contact, and is inversely proportional to the water film thickness. Note that, since the amount of dissolved oxygen that can react on the surface of the metal structure is limited, the corrosion rate does not infinitely become large. Therefore, the temporal change in the corrosion rate due to the change in the water film thickness (decrease in the water film thickness) exhibits a behavior as illustrated in FIG. 9.

As described above, there are the influence of the wet area and the influence of the water film thickness on the corrosion rate, but the actual corrosion rate changes in a form in which these influences are simultaneously taken into consideration.

In embodiments of the present invention, as described below, first, the influence of the wet area on the corrosion rate is expressed as a damping function, the influence of the water film thickness on the corrosion rate is expressed as an increase function, and a function expressed by a product of these functions is used as a model representing the corrosion rate of metal in soil.

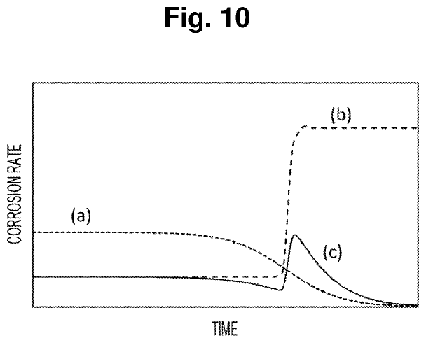

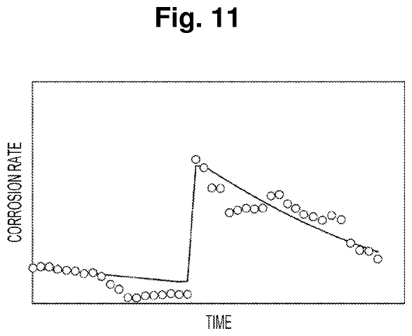

FIG. 10 illustrates an example of a temporal change model of the corrosion rate of metal in soil. In FIG. 10, (a) illustrates the change in the wet area, (b) illustrates the change in the corrosion rate due to (derived from) the thickness of the water film, and (c) illustrates the change in the corrosion rate of the metal structure buried in soil. Further, FIG. 11 is an example of fitting the above-described corrosion rate model (solid line) to the measured values (white circles) illustrated in FIG. 5. As described above, it is known that the model of the corrosion rate well explains a tendency of the corrosion rate of the soil actually measured by the alternating current impedance method.

Note that it is known that both the influence of the wet area and the influence of the water film thickness are determined by a solid phase present on the surface of the metal structure, that is, the state of the soil particles. For example, when there is soil having a large and uniform particle size, drainage is fast and the capillary phenomenon is difficult to work, so that the wet area decreases quickly and the state of the thin water film is not maintained for a long time.

On the other hand, in a case of soil having a small particle diameter like clay, drainage is slow and the decrease in the wet area is extremely slow. However, as the water escapes, the thin water film state is likely to be formed on the surface of the metal structure with slight water retained by the capillary phenomenon.

As described above, the influence of the wet area and the influence of the water film thickness are not independent from each other, but they are determined by the state of soil particles and are deeply related to each other. Therefore, if the damping function that expresses the temporal change in the wet area is known, the increase function that expresses the temporal change in the water film thickness can also be defined. Therefore, it is only necessary to know the temporal change in the wet area to know the temporal change in the corrosion rate.

Therefore, if the information of the temporal change in the soil water content in one rain is obtained, the temporal change model of the corrosion rate can be derived on the basis of the information. Therefore, thereafter, it becomes possible to obtain (estimate) the corrosion rate only from the information of the soil water content by using this model.

Next, estimation of the damping function representing the temporal change in the ratio of the wet area based on the measured water content measurement value will be described. This estimation can be performed by deriving the damping function to be best fitted using the temporal change in the soil water content as it is. The function to be fitted is not particularly limited, but for example, a following damping function Y1 can be used. Note that, in the following formulas, t is time, and a, b, and t1 are parameters.

$$Y 1 = \{ \frac{1}{1 + a \cdot {e}^{b ( t - t 1 )}} \} \qquad Equation 1$$

Note that the soil water content and the wet area ratio on the surface of the metal structure are not strictly the same. Therefore, for example, a relationship between the soil water content and the wet area ratio on the surface of the metal structure may be derived in advance by the following method, and the temporal change in the wet area ratio on the surface of the metal structure may be estimated from the measurement result of the soil water content.

First, a soil moisture sensor and an electrode of the alternating current impedance method are buried in the soil, and changes in the water content and the corrosion rate associated with dry and wet conditions of the soil are measured.

Next, it is possible to derive a relationship between the damping function and the water content by fitting the change in the corrosion rate with the temporal change function of the corrosion rate given by the product of the damping function and the rate increase function and comparing the changes in the water content and the damping function.

The temporal change in the wet area ratio on the surface of the metal structure is derived from the measurement result of the soil water content on the basis of the relationship obtained in advance. A function used for fitting the derived temporal change in the wet area ratio on the surface of the metal structure is not particularly limited, but the above-described damping function Y1 can be used.

Next, estimation of the rate increase function using the damping function will be described. The rate increase function derived from the water film thickness on the surface of the metal structure is estimated from the damping function obtained (estimated) as described above. As described above, the influence of the wet area and the influence of the water film thickness are not independent from each other, but they are determined by the state of soil particles and are deeply related to each other. Therefore, if the damping function that expresses the temporal change in the wet area is known, the increase function that expresses the temporal change in the water film thickness can also be defined. Therefore, it is only necessary to know the temporal change in the wet area to know the temporal change in the corrosion rate.

That is, by deriving the relationship between the damping function expressing the temporal change in the wet area and the increase function expressing the temporal change in the water film thickness in advance by an experiment or the like, it is possible to estimate the rate increase function derived from the water film thickness on the surface of the metal structure from the estimated damping function. Note that the rate increase function is not particularly limited, and for example, a following function Y2 is one of the functions that can well describe the phenomenon. Note that K, α, β, γ, and t₀ are parameters.

$$Y 2 = \{ \frac{α \cdot K \cdot {e}^{β ( t - {t}_{0} )}}{( K - α ) + {e}^{β ( t - {t}_{0} )}} + γ \} \qquad Equation 2$$

Next, estimation of the temporal change function of the corrosion rate will be described. The temporal change function of the corrosion rate given by the product of the damping function and the rate increase function is estimated. In a case where Y1 is used as the damping function and Y2 is used as the rate increase function, the temporal change in the estimated corrosion rate is expressed by a following function Y.

$$Y = \{ \frac{α \cdot K \cdot {e}^{β ( t - {t}_{0} )}}{( K - α ) + {e}^{β ( t - {t}_{0} )}} + γ \} \times \{ \frac{1}{1 + a \cdot {e}^{b ( t - t 1 )}} \} \qquad Equation 3$$

As described above, through the above-described steps, the information of the temporal change in the corrosion rate can be estimated from the information of the temporal change in the soil water content. Therefore, when the corrosion rate is desired to be known, it can be estimated only by measuring the soil water content without using a conventional expensive alternating current impedance device or the like.

As described above, according to embodiments of the present invention, the damping function is estimated on the basis of the water content measurement value, the rate increase function derived from the thickness of the water film is estimated from the damping function, and the temporal change function of the corrosion rate given by the product of the damping function and the rate increase function is estimated, so that the corrosion rate of the metal buried in the ground becomes able to be easily and inexpensively measured.

Note that the present invention is not limited to the embodiment described above, and it is obvious that many modifications and combinations can be made by a person having ordinary knowledge in the art within the technical idea of the present invention.

### REFERENCE SIGNS LIST

101 Sensor
102 First processing circuit
103 Second processing circuit
104 Third processing circuit
105 Fourth processing circuit
106 Storage unit
107 Display unit

1-4. (canceled)

5. A corrosion estimation method comprising:
a measurement step of measuring a water content of soil of a target land;
a first processing step of estimating a damping function representing a temporal change in a ratio of an area wetted with water on a surface of a metal structure buried in the target land based the water content measured in the measurement step;
a second processing step of estimating a rate increase function that increases an estimated corrosion rate of the metal structure, the rate increase function estimated based on the damping function and a thickness of a water film formed on the surface of the metal structure; and
a third processing step of estimating a temporal change function of the estimated corrosion rate based on a product of the damping function and the rate increase function.

6. The corrosion estimation method according to claim 5, further comprising:
a fourth processing step of obtaining the estimated corrosion rate of the metal structure from the measured water content of the soil of the target land based on the temporal change function.

7. The corrosion estimation method according to claim 5, wherein the damping function is presented by:
$$Y 1 = \{ \frac{1}{1 + a \cdot {e}^{b ( t - t 1 )}} \} ,$$
wherein Y1 is the damping function, t is time, and a, b, and t1 are constants.

8. The corrosion estimation method according to claim 7, wherein the rate increase function is represented by:
$$Y 2 = \{ \frac{α \cdot K \cdot {e}^{β ( t - {t}_{0} )}}{( K - α ) + {e}^{β ( t - {t}_{0} )}} + γ \} ,$$
wherein Y2 is the rate increase function, and K, α, β, γ, and t₀ are constants.

9. The corrosion estimation method according to claim 8, wherein the temporal change function is represented by:
$$Y = \{ \frac{α \cdot K \cdot {e}^{β ( t - {t}_{0} )}}{( K - α ) + {e}^{β ( t - {t}_{0} )}} + γ \} \times \{ \frac{1}{1 + a \cdot {e}^{b ( t - t 1 )}} \} ,$$
wherein Y is the temporal change function.

10. A corrosion estimation device comprising:
a sensor configured to measure a water content of soil of a target land;
a storage device comprising instructions; and
one or more processors in communication with the storage device, wherein the one or more processors execute the instructions to:
estimate a damping function representing a temporal change in a ratio of an area wetted with water on a surface of a metal structure buried in the target land based on the water content measured by the sensor;
estimate a rate increase function that increases an estimated corrosion rate of the metal structure, the rate increase function being derived based on the damping function and a thickness of a water film on the surface of the metal structure; and
estimate a temporal change function of the estimated corrosion rate based on a product of the damping function and the rate increase function.

11. The corrosion estimation device according to claim 10, wherein the one or more processors execute the instructions to further:
obtain the estimated corrosion rate of the metal structure from the measured water content of the soil of the target land based on the temporal change function.

12. The corrosion estimation device according to claim 10, wherein the damping function is presented by:
$$Y 1 = \{ \frac{1}{1 + a \cdot {e}^{b ( t - t 1 )}} \} ,$$
wherein Y1 is the damping function, t is time, and a, b, and t1 are constants.

13. The corrosion estimation device according to claim 12, wherein the rate increase function is represented by:
$$Y 2 = \{ \frac{α \cdot K \cdot {e}^{β ( t - {t}_{0} )}}{( K - α ) + {e}^{β ( t - {t}_{0} )}} + γ \} ,$$
wherein Y2 is the rate increase function, and K, α, β, γ, and t₀ are constants.

14. The corrosion estimation device according to claim 13, wherein the temporal change function is represented by:
$$Y = \{ \frac{α \cdot K \cdot {e}^{β ( t - {t}_{0} )}}{( K - α ) + {e}^{β ( t - {t}_{0} )}} + γ \} \times \{ \frac{1}{1 + a \cdot {e}^{b ( t - t 1 )}} \} ,$$
wherein Y is the temporal change function.
